(19) United States
(12) Patent Application Publication
(10) Pub. No.: US 2017/0209693 A1
(43) Pub. Date: Jul. 27, 2017

(54) TRANSCUTANEOUS ELECTRICAL NERVE STIMULATION (TENS) APPARATUS

(71) Applicant: M.I.Tech Co., Ltd., Gyeonggi-do (KR)

(72) Inventors: Soon Kwan An, Seoul (KR); Jeong U Lim, Gyeonggi-do (KR); Chan Hee Kim, Seoul (KR); Sang Hyun Kim, Seoul (KR); Chang Sik Mun, Gyeonggi-do (KR)

(73) Assignee: M.I.Tech Co., Ltd., Gyeonggi-do (KR)

(21) Appl. No.: 15/321,669

(22) PCT Filed: Apr. 22, 2015
(86) PCT No.: PCT/KR2015/003995

§ 371 Date: Dec. 22, 2016

(30) Foreign Application Priority Data
Jun. 25, 2014 (KR) 10-2014-0078291

(51) Int. Cl. A61N 1/36; A61N 1/04

(52) U.S. Cl. CPC A61N 1/36014; A61N 1/0456; A61N 1/0492; A61N 1/0476; A61N 1/048; A61N 1/36021

(57) ABSTRACT

Provided is a transcutaneous electrical nerve stimulation (TENS) apparatus, which includes an electric signal generator configured to generate an electrical stimulation signal, an electrode configured to transmit an electrical stimulation corresponding to the electrical stimulation signal generated by the electric signal generator to a user, and a connection unit configured to connect the electric signal generator to the electrode, wherein the electrode includes a pair of annular contact points for an electrical connection with the electric signal generator. Here, the connection unit may include magnetic material.

### TECHNICAL FIELD

The present disclosure relates to a transcutaneous electrical nerve stimulation (TENS) apparatus, and more particularly, to a TENS apparatus to/from which an electrode may be attached or detached.

### BACKGROUND ART

Recently, various transcutaneous electrical nerve stimulation (TENS) apparatuses for relieving pain by persistently applying electrical stimulations to nervus antennalis to restrain the sense of pain have been proposed.

However, in an existing TENS apparatus, an electric signal generator for generating an electrical stimulation signal and an electrode for providing an electrical stimulation corresponding to the generated electrical stimulation signal to a user are integrally combined, or a stimulator is connected to an electrode using a cable. However, if the existing TENS apparatus is attached to a portion of a human body which is not reached by the hand of the user, it is difficult to control the TENS apparatus, and the cable causes inconvenience in use.

### RELATED LITERATURES

### Patent Literature

KR 10-1364598

### DISCLOSURE OF INVENTION

### Technical Problem

The present disclosure is directed to providing a TENS apparatus in which an electric signal generator and an electrode may be attached or detached to/from each other for the convenience of a user.

### Solution to Problem

In one general aspect, the present disclosure provides a transcutaneous electrical nerve stimulation (TENS) apparatus, which includes: an electric signal generator configured to generate an electrical stimulation signal; an electrode configured to transmit an electrical stimulation corresponding to the electrical stimulation signal generated by the electric signal generator to a user; and a connection unit configured to connect the electric signal generator to the electrode, wherein the electrode includes a pair of annular contact points for an electrical connection with the electric signal generator.

In an embodiment, the annular contact point may be located at a center of an upper surface of the electrode. In another embodiment, the annular contact point may be located at one side of an upper surface of the electrode.

In an embodiment, the connection unit may include magnetic material.

In an embodiment, the electric signal generator may include a pair of output terminals located at a lower surface of a housing to output the electrical stimulation signal.

In an embodiment, the electric signal generator may further include an external input terminal located at one side of the housing to receive an input from an external object.

In an embodiment, the electric signal generator may include: a signal generation unit configured to generate the electrical stimulation signal; and a control unit configured to control each signal generation unit of the electric signal generator.

In an embodiment, the electric signal generator may further include a communication unit configured to communicate with an external device, in the housing.

In an embodiment, the electric signal generator may be reusable.

In an embodiment, the electrode may include: a body; and a pair of electrode pads located at a lower surface of the body to transmit the electrical stimulation signal to a user.

In an embodiment, the electrode may further include an anti-separation frame configured to preventing the electrode connected to the electric signal generator from being separated therefrom, and the anti-separation frame may surround the pair of annular contact points.

In an embodiment, the electrode may be replaceable.

In an embodiment, the electrode may further include: a circuit board at which the pair of annular contact points are formed; and a conductive connection unit connected to the pair of annular contact points to transmit each electrical stimulation signal to the pair of electrode pads.

In an embodiment, the body may be made of a circuit board at which the annular contact points are formed.

In an embodiment, the electrode pad may be made of a conductive hydrogel pad which allows the electrode to be attached to the skin of the user.

In an embodiment, the conductive hydrogel pad may be removable and replaceable.

### Advantageous Effects of Invention

According to the present disclosure, the TENS apparatus may use various kinds of electrodes since an electric signal generator and an electrode may be attached or detached to/from each other.

According to the present disclosure, since the TENS apparatus may be connected to an electrode without using a separate connection cable, the electric signal generator may be easily attached or detached, and the detached electric signal generator may be easily attached again after an operation mode or the like is adjusted.

In addition, according to the present disclosure, since the TENS apparatus may be electrically connected to an electrode through an annular contact point, it may be electrically connected to the electrode regardless of a rotating direction of the electric signal generator.

### BRIEF DESCRIPTION OF DRAWINGS

FIG. 1 shows a TENS apparatus according to an embodiment of the present disclosure.

FIG. 2 shows an electric signal generator according to an embodiment of the present disclosure.

FIG. 3 shows an inner configuration of the electric signal generator according to an embodiment of the present disclosure.

FIG. 4 shows an electrode according to an embodiment of the present disclosure.

FIG. 5 shows an electrode according to another embodiment of the present disclosure.

FIG. 6 shows an electrode according to another embodiment of the present disclosure.

### BEST MODE FOR CARRYING OUT THE INVENTION

Exemplary embodiments now will be described more fully hereinafter with reference to the accompanying drawings, in which exemplary embodiments are shown. The present disclosure may, however, be embodied in many different forms and should not be construed as limited to the exemplary embodiments set forth therein.

The terms used in the present disclosure are selected as most common terms generally used in the art, which however may differ as intended by those skilled in the art or according to custom or new technologies. In addition, in special cases, a term arbitrarily chosen by the applicant can be used, and in this case, its meaning will be explained in a suitable portion of this specification. Therefore, the terms used in the present disclosure should be interpreted based on substantial meanings of the terms and overall contents of the present disclosure, without being limited to the expressions of the terms.

In the present disclosure, a transcutaneous electrical nerve stimulation (TENS) apparatus means an apparatus for providing an electrical stimulation corresponding to an electrical stimulation signal generated by a stimulator to a user through an electrode. For example, the TENS apparatus may be a low-frequency medical apparatus, an electrical stimulation apparatus or a portable electrical stimulation apparatus, which provides an electrical stimulation to the skin of a user for medical treatment, pain relief, vomit or nausea relief, morning sickness relief or the like.

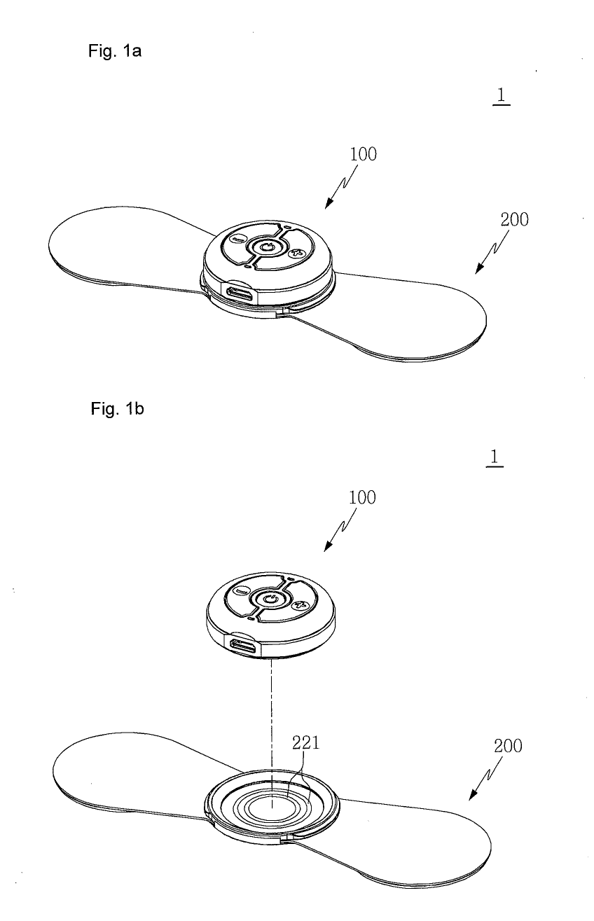

FIG. 1 shows a TENS apparatus according to an embodiment of the present disclosure. In more detail, FIG. 1a shows a TENS apparatus 1 in which an electric signal generator 100 is connected to an electrode 200, and FIG. 1b shows a TENS apparatus 1 in which the electric signal generator 100 is separated from the electrode 200.

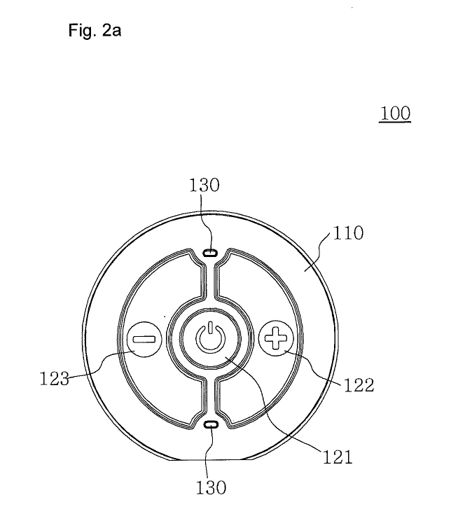

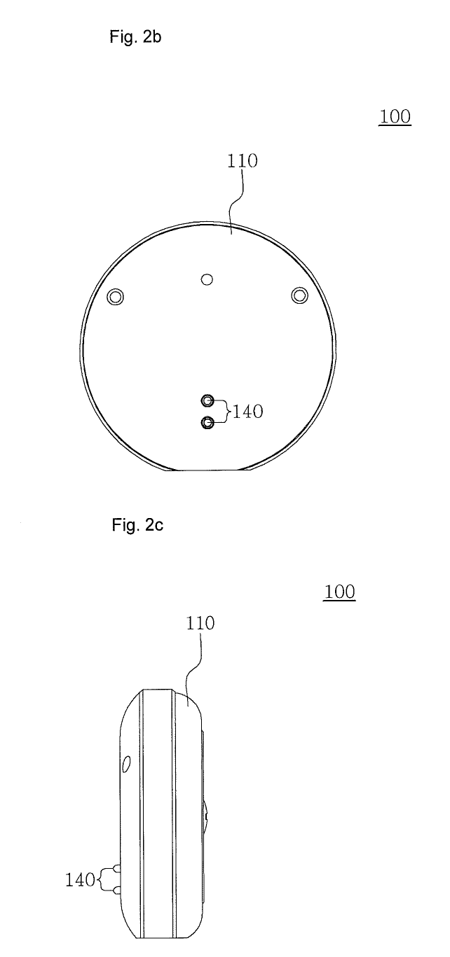

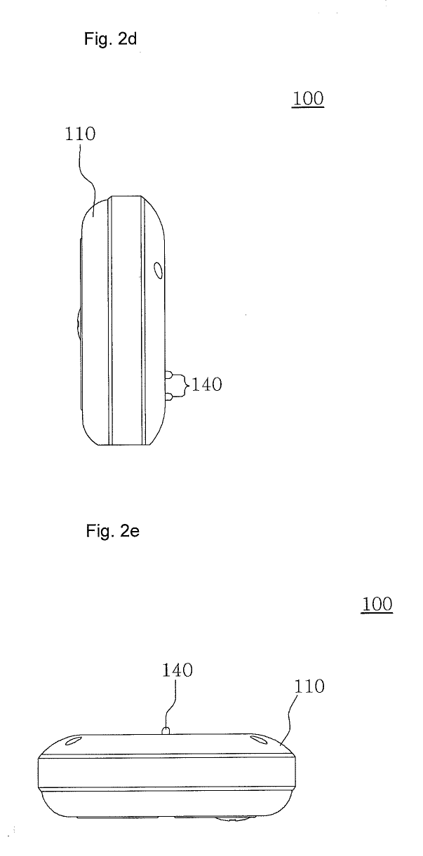

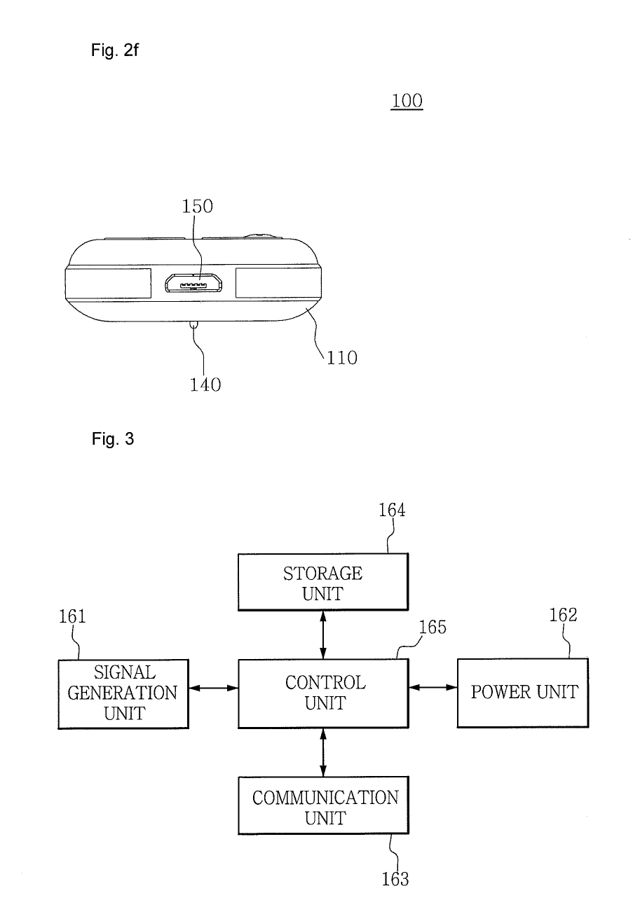

Referring to FIGS. 1a and 1b, the TENS apparatus 1 may include an electric signal generator 100 and an electrode 200. Here, the electric signal generator 100 means a portion which generates an electrical stimulation signal, and may also be called a stimulator. In an embodiment, the electric signal generator 100 may be a reusable part. The electric signal generator 100 will be described in detail later with reference to FIGS. 2 and 3.

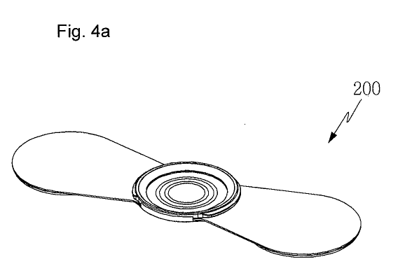

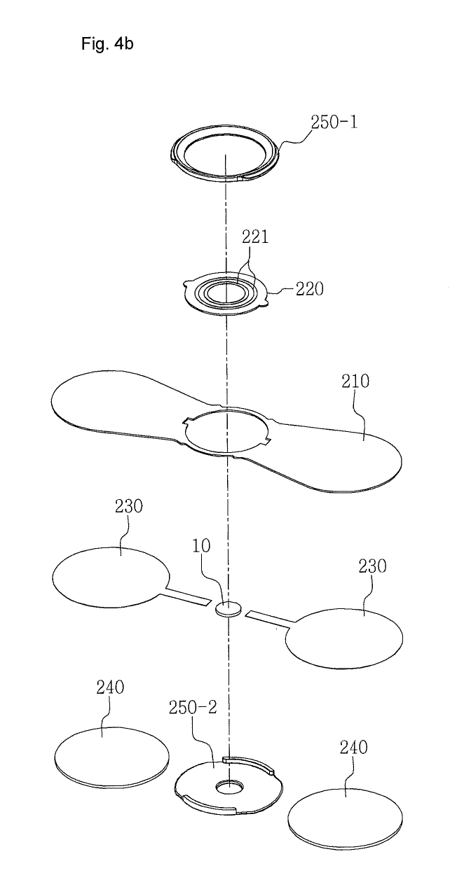

Here, the electrode 200 means a portion which provides an electrical stimulation corresponding to an electrical stimulation signal generated by the electric signal generator 100 to a user. In an embodiment, the electrode 200 may be a replaceable part. The electrode 100 will be described in detail later with reference to FIGS. 4 to 6.

The electric signal generator 100 and the electrode 200 may be connected to each other through a connection unit. Here, the connection unit may be made of material capable of repeatedly attaching or detaching the electric signal generator 100 and the electrode 200 to/from each other. In an embodiment, the connection unit may be made of magnetic material. For example, a first magnet in the electric signal generator 100 and a second magnet in the electrode 200, which have opposite polarities, may be used as the connection unit. In this case, the electric signal generator 100 and the electrode 200 may be connected to each other by means of a magnetic force (for example, an attractive force). By doing so, the electric signal generator 100 may be connected to the electrode 200 without any separate connection cable, and thus the user may easily attach or detach the electric signal generator 100. In another embodiment, the connection unit may be made of Velcro, or other adhesive material. In addition, the connection unit may be made of a connection cable.

In an embodiment, the electrode 200 may include a pair of annular contact points 221 for making an electrical connection with the electric signal generator 100. In an embodiment, the pair of annular contact point 221 may have different radii. If the electric signal generator 100 is electrically connected to the electrode 200 by using such an annular contact point, the electric signal generator 100 and the electrode 200 may be electrically connected to each other regardless of a rotating direction of the electric signal generator 100. By doing so, the user may easily detach the stimulator 100, and after adjusting an operation mode or the like of the stimulator 100 in a detached state, the user may easily attach the stimulator 100 again to the electrode 200.

FIG. 2 is a diagram showing an appearance of an electric signal generator according to an embodiment of the present disclosure. In more detail, FIG. 2a is a front view showing the electric signal generator 100, FIG. 2b is a rear view showing the electric signal generator 100, FIG. 2c is a left side view showing the electric signal generator 100, FIG. 2d is a right side view showing the electric signal generator 100, FIG. 2e is a plane view showing the electric signal generator 100, and FIG. 2f is a bottom view showing the electric signal generator 100.

Referring to FIGS. 2a to 2f, the electric signal generator 100 may include an input unit, a display unit and an output unit out of the housing 110. Here, the input unit and the display unit may give an interaction between the electric signal generator 100 and the user and thus may be called a user interface. In addition, the electric signal generator 100 may further include an external input terminal 150 as an optional component.

The input unit may detect a control input of the user. As shown in FIG. 2a, the input unit may include at least one control button. Here, the control button means a button for controlling an operation of the electric signal generator 100.

The control button may include a power/mode button 121 (hereinafter, referred to as a power button 121) for turning on/off the electric signal generator 100 and/or controlling an operation mode of the electric signal generator 100. Here, the operation mode may include at least one mode. For example, the operation mode may include at least one of a tapping mode, a massaging mode, a strong massaging mode and a combination mode. In addition, one of the operation modes may be set as a basic mode. For example, the tapping mode may be set as a basic mode.

Here, the tapping mode represents a mode in which an electrical stimulation for tapping a stimulation spot is given to the user. In an embodiment, the tapping mode may have a stimulation waveform in which an ascending pulse and a descending pulse with preset frequencies and intensities are alternately provided repeatedly. In this case, a pulse interval between the ascending pulse and the descending pulse may be 1 s, and a pulse width of the ascending pulse and the descending pulse may be 110 μs, without being limited thereto.

Here, the massaging mode represents a mode in which an electrical stimulation for massaging a stimulation spot is given to the user. In an embodiment, the massaging mode may have a stimulation waveform in which a process of continuously providing an ascending pulse during a preset first duration time, giving a first interval, and then continuously providing a descending pulse during a preset first duration time is repeated. In this case, the first duration time may be 6.5 s, and the first interval may be 1.5 s, without being limited thereto. In addition, the ascending pulse and the descending pulse may have a pulse width of 70 μs, and a gap between the pulses may be 60 ms, without being limited thereto.

Here, the strong massaging mode represents a mode in which an electrical stimulation for massaging a stimulation spot more strongly in comparison to the massaging mode is given to the user. In an embodiment, the strong massaging mode may have a stimulation waveform in which a process of continuously providing an ascending pulse for a preset second duration time, giving a second interval, and continuously providing a descending pulse for a preset second duration time is repeated. In this case, the second duration time may be 4 s, and the second interval may be 2 s, without being limited thereto. In addition, the ascending pulse and the descending pulse may have a pulse width of 60 μs, and a gap between the pulses may be 30 ms, without being limited thereto.

Here, the combination mode represents a mode in which electrical stimulations corresponding to the tapping mode, the massaging mode and the strong massaging mode are sequentially provided to the user repeatedly.

In an embodiment, if the power button 121 is pressed over a preset time (for example, 3 seconds), the electric signal generator 100 may turn on or off. If turning on, the electric signal generator 100 may operate in the basic mode. For example, if turning on, the electric signal generator 100 may operate in the tapping mode which is the basic mode. In an embodiment, in a turning-on state, if the power button 121 is pressed shortly (for example, less than 1 second), the operation mode of the electric signal generator 100 may vary in order, without being limited thereto. For example, if the power button 121 is pressed shortly in the tapping mode, the operation mode of the electric signal generator 100 may be changed from the tapping mode to the combination mode sequentially according to a preset order. In addition, the control button may further include an increase button 122 for increasing stimulation strength and a decrease button 123 for decreasing stimulation strength.

The power button 121, the increase button 122 and the decrease button 123 may be provided at various locations of the housing 110. For example, the power button 121, the increase button 122 and the decrease button 123 may be provided at an upper side of the housing 110. In another example, the power button 121 may be provided at one side of the housing 110, and the increase button 122 and the decrease button 123 may be provided at an upper side of the housing 110.

The display unit may display a state of the electric signal generator 100. The display unit may be an optional component of the electric signal generator 100. In an embodiment, as shown in FIG. 2a, the display unit may include at least one indicator light 130 for displaying a state of the electric signal generator 100. For example, the display unit may include a first indicator light and a second indicator light. Here, each indicator light may include at least one LED. For example, each indicator light may include at least one of yellow, green, sky-blue and violet LEDs. In this case, the electric signal generator 100 may provide state information about the electric signal generator 100 to the user by using at least one indicator light. For example, the electric signal generator may provide information about the operation mode to the user by using the first indicator light and provide information about the operation state to the user by using the second indicator light. In an embodiment, as shown in FIG. 2a, the indicator light 130 may be located at an upper side of the housing 110.

Here, the state information may include information about at least one of an operation state, an operation mode and an operation strength. In an embodiment, the operation state may include at least one of a normal operation state, a sleep state and a charging state. Here, the normal operation state means that the electric signal generator 100 normally operate, the sleep state means that the electric signal generator 100 is in a sleep or standby state, and the charging state means that the electric signal generator 100 is being charged with power.

The output unit may output an electrical stimulation signal. In an embodiment, as shown in FIGS. 2b to 2f, the electric signal generator 100 may include a pair of output terminals 140 for outputting an electrical stimulation signal. For example, the output unit 130 may include a pair of positive (+) and negative (−) terminals for outputting an electrical stimulation signal.

In an embodiment, the output terminal 140 may be located at a lower surface of the housing 110. For example, the output terminal 140 may be a pair of terminals located at different distances from the center of the lower surface of the housing 110 so as to be electrically connected to a pair of annular contact points with different radii, respectively. In other words, for electric connection between a pair of output terminals and a pair of annular contact points, a distance from a center of each output terminal may be identical to a radius of each annular contact point. In this case, a pair of output terminals in the electric signal generator may be electrically connected to a pair of annular contact points in the electrode, respectively. By doing so, the electric signal generator 100 may be electrically connected to the electrode. In this case, the TENS apparatus may provide an electrical stimulation generated by the electric signal generator 100 to the user through the electrode by electrically connecting each output terminal of the electric signal generator 100 to each annular contact point of the electrode in a state where the electric signal generator 100 is attached to the electrode through the connection unit.

As shown in FIG. 2f, the external input terminal 150 is an optional component, which represents a terminal for receiving an input from an external object. In an embodiment, the external input terminal 150 may receive power from an external power source to charge the electric signal generator 100 with power. In another embodiment, the external input terminal 150 may receive setting information about the operation mode of the electric signal generator 100 from the external device.

FIG. 3 shows an inner configuration of the electric signal generator.

Referring to FIG. 3, the electric signal generator 100 may include a signal generation unit 161, a power unit 162, a communication unit 163, a storage unit 164 and a control unit 165 in the housing 110. Here, the communication unit 163 and the storage unit 164 may be optional components.

The signal generation unit 161 may generate an electrical stimulation signal. The signal generation unit 161 may generate an electrical stimulation signal corresponding to the operation mode and the operation strength. In more detail, the signal generation unit 161 may generate an electrical stimulation signal with stimulation waveform corresponding to the operation mode and stimulation strength corresponding to the operation strength. In an embodiment, the electrical stimulation signal may be a low-frequency pulse signal. In an embodiment, the signal generation unit 161 may be activated when the TENS apparatus is attached to the skin of the user. For example, the signal generation unit 161 may be activated to generate an electrical stimulation signal only when the electric signal generator 100 is connected to the electrode 200 and the electrode 200 is attached to the skin of the user.

The power unit 162 may supply power to each component of the electric signal generator 100. In an embodiment, the power unit 162 may include at least one rechargeable or replaceable battery. If the power unit 162 includes a rechargeable battery, the electric signal generator 100 may charge the battery of the power unit 162 with the power supplied from the external power source to the external input terminal.

The communication unit 163 may communicate with an external device. In an embodiment, the communication unit 163 may communicate with an external device by using at least one wire/wireless communication method. For example, the communication unit 163 may communicate with an external device wirelessly by means of bluetooth communication, without being limited thereto. In an embodiment, the external device may be a remote controller or a mobile phone (for example, a smart phone) having an application for controlling the electric signal generator 100. In this case, an external device may be paired with the TENS apparatus through the communication unit 163, and the external device paired with the electrical stimulation apparatus 1 may control the electric signal generator 100 through a communication signal (or, a control signal). Here, the communication signal may be a bluetooth signal, a RF signal, or any other kinds of signals.

The storage unit 164 may store data. In an embodiment, the storage unit 164 may store preset data about the operation mode of the electric signal generator 100. In another embodiment, the storage unit 164 may store data about the operation mode of the electric signal generator 100, input through the external input terminal. In this case, the user may newly update information about the operation mode of the electric signal generator 100.

The control unit 165 may control at least one component provided at the electric signal generator 100. In more detail, the control unit 165 may control each unit described above and also control data transmission and/or reception between units.

The control unit 165 may detect a control input of the user through the input unit. In addition, the control unit 165 may determine the operation mode and the operation strength based on the detected control input. In addition, the control unit 165 may issue an electrical stimulation signal corresponding to the determined operation mode and operation strength.

In addition, the control unit 165 may determine a state (or, the operation state) of the electric signal generator 100. Here, the state of the electric signal generator 100 may include a first state in which the electrode 200 is electrically connected to the electric signal generator 100 and a second state in which the electrode 200 is not electrically connected to the electric signal generator 100. When the electric signal generator 100 is in the first state, the control unit 165 may provide an electrical stimulation corresponding to the electrical stimulation signal to the user through the electrode. By doing so, stimulation treatment or the like may be provided to the user. In addition, When the electric signal generator 100 is in the second state, the control unit 165 may discharge a residual charge of the electric signal generator 100 according to a preset period. By doing so, it is possible to prevent a large amount of charges from being transferred unexpectedly, when an electrical stimulation is provided to the user, and thus it is possible to provide safe electrical stimulation to the user.

In addition, the control unit 165 may detect the change of state of the electric signal generator 100. When the state of the electric signal generator 100 changes from the second state to the first state, the control unit 165 may change stimulation strength of the electrical stimulation signal into a preset basic strength. In an embodiment, the basic strength may be a stimulation strength lower than the operation strength. In addition, the control unit 165 may enhance stimulation strength step by step from the basic strength to the operation strength. In the present disclosure, this may be called a soft restart function, which is one of safety functions.

In addition, if a preset sleep condition is satisfied, the control unit 165 may discharge a residual charge of the electric signal generator 100 and keep the electric signal generator 100 in the sleep state. Here, the case of satisfying the sleep condition may include at least one of a case where a residual power of the electric signal generator 100 is lower than a preset level, a case where an electrical stimulation signal is not output during a preset first time and a case where an electrical stimulation signal is output during a preset second time. In addition, the control unit 165 may notify the state of the electric signal generator 100 to the user through the display unit.

In addition, the control unit 165 may control the TENS apparatus 1 based on a control signal received from an external device through the communication unit 163. FIG. 3 is a block diagram according to an embodiment of the present disclosure, in which blocks separately depicted represent logically-distinguished elements of a device. Therefore, the elements of the device may be mounted as a single chip or multiple chips depending on the device design.

Hereinafter, if each step or operation performed by the electric signal generator 100 or the TENS apparatus 1 is initiated or progressed by a received user input, it should be understood that a process of generating and receiving a signal according to the received user input is included in the above procedure, even though it is not described in detail here. Hereinafter, it may be expressed that the control unit 165 controls at least one unit included in the electric signal generator 100 or the TENS apparatus 1 according to an input, and the control unit 165 may also be described as being identical to the electric signal generator 100 and the TENS apparatus 1.

FIG. 4 shows an electrode according to an embodiment of the present disclosure. In more detail, FIG. 4a shows an appearance of the electrode 200 having a basic form, and FIG. 4b shows detailed configuration of the electrode 200.

Referring to FIGS. 4a and 4b, the electrode 200 may include a body 210, a circuit board 220, a conductive connection unit 230 and a pair of electrode pads 240. In addition, the electrode 200 may include at least one anti-separation frame in order to prevent the electric signal generator 100 and the electrode 200, connected (attached) to each other, from being easily separated. In an embodiment, the anti-separation frame may have a cradle form. In an embodiment, the anti-separation frame may surround the annular contact point 221. In an embodiment, the anti-separation frame configured as above may be made of fiber-reinforced plastic, ceramic, silicon, sapphire glass, soda glass or the like. As shown in FIG. 4b, the anti-separation frame may be composed of an upper frame 250-1 having a rim structure and a lower frame 250-2 having at least one groove at which a magnet 10 serving as the connection unit is located.

In an embodiment, the body 210 may be made of silicon rubber, polyurethane or the like. In this case, the electrode 200 may be bent into various shapes, and thus the user may attach the electrode 200 to portions with various curves.

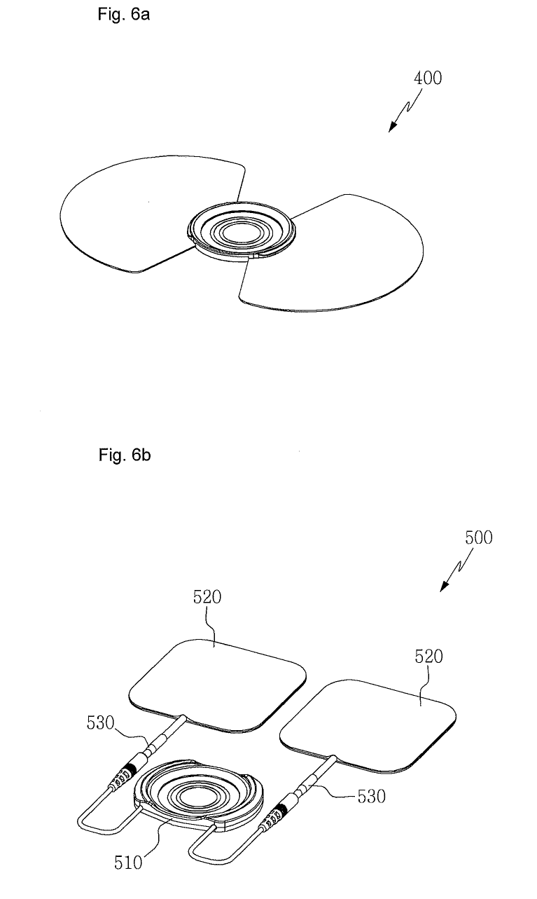

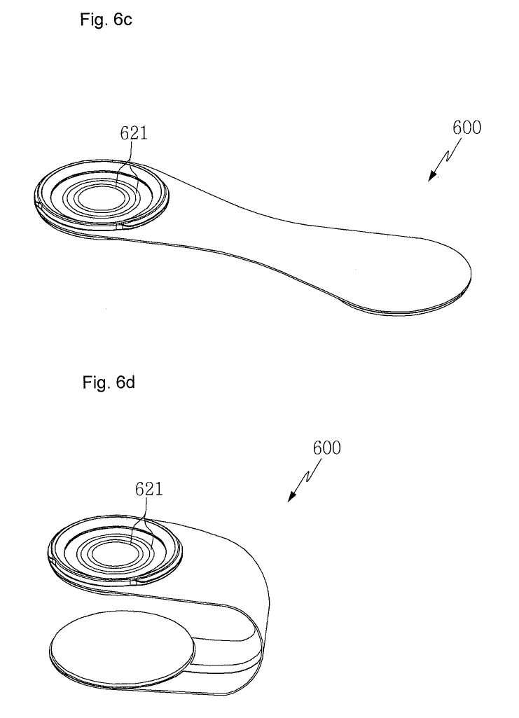

The circuit board 220 may include a pair of annular contact points 221. In an embodiment, the circuit board 220 may be a printed circuit board (PCB) at which a pair of annular contact points 221 are formed. In an embodiment, the annular contact point may be located at the center of the upper surface of the body 210, without being limited thereto. This will be described later in detail with reference to FIG. 6c.

The conductive connection unit 230 may be respectively connected to the pair of annular contact points to transmit an electrical stimulation signal to each of the pair of electrode pads 240. In an embodiment, the conductive connection unit 230 may be conductive sheet, conductive textile, conductive ink printing or the like.

The electrode pad 240 may transmit an electrical stimulation signal to the user. For example, the electrode pad 240 may be composed of a conductive hydrogel pad attached to the skin of the user. In an embodiment, the electrode pad 240 may be located at a bottom surface of the body 210. In an embodiment, the electrode 200 may have an electrode pad 240 with various sizes and shapes. The electrode pad 240 with various sizes and shapes will be described later in detail with reference to FIG. 6.

As described above, the electrode 200 may be connected (attached) to the electric signal generator 100 through the connection unit. In this case, the electrode 200 and the electric signal generator 100 may be electrically connected to each other by making an electric contact between each annular contact point of the electrode 200 and each output terminal of the electric signal generator 100. By doing so, the electrical stimulation signal generated by the electric signal generator 100 may be transmitted to the electrode 200. In addition, each electrical stimulation signal transmitted through the pair of annular contact points may be transmitted to the pair of electrode pads 240, respectively, through the conductive connection unit 230. By doing so, an electrical stimulation corresponding to the electrical stimulation signal generated by the electric signal generator 100 may be transmitted to the skin of the user through the electrode pad 240 of the electrode 200.

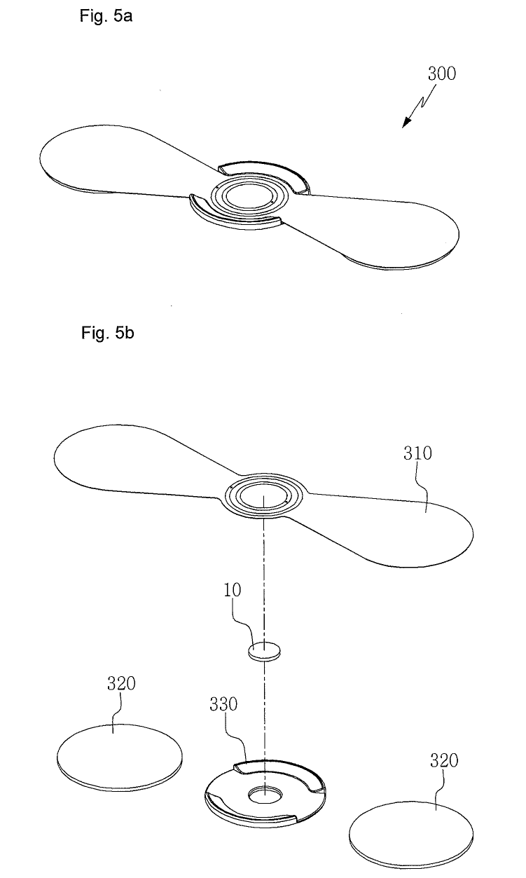

FIG. 5 shows an electrode according to another embodiment of the present disclosure. In more detail, FIG. 5a shows an appearance of the electrode 300 having a circuit board form, and FIG. 5b shows detailed configuration of the electrode 300.

Referring to FIGS. 5a and 5b, the electrode 300 may include a body 310 having a circuit board form and a pair of electrode pads 320. Here, the electrode pad 320 has already described above with reference to FIG. 4 and thus is not described here in detail. In addition, the electrode 300 may include an anti-separation frame 330 having a cradle shape in order to prevent the electric signal generator 100 and the electrode 200, connected to each other, from being easily separated. However, the anti-separation frame of FIG. 5 has a rim structure, different from the anti-separation frame of FIG. 4, and may be composed of just a single lower frame 330 having at least one groove at which a magnet serving as the connection unit 10 is located.

The body 310 of FIG. 5 may be configured so that the body 310 is a single circuit board, different from the body 210 of FIG. 4. In an embodiment, the circuit board may be a board where a pair of annular contact points are formed. For example, the circuit board may be a board at which the annular contact point is formed at the center of the upper surface of the body. In an embodiment, the circuit board may be made of polyimide, liquid crystal polymer film, PET or the like. In an embodiment, the circuit board may include conductor such as thermally-deposited gold, plasma, or the like. By doing so, the electrode 300 of FIG. 5 may transmit an electrical stimulation signal from the annular contact point to the electrode pad 320 without any separate conductive connection unit 230, different from the electrode 200 of FIG. 4.

FIG. 6 shows an electrode according to another embodiment of the present disclosure. As shown in FIGS. 6a to 6d, the electrode may have various sizes, shapes and kinds.

Referring to FIG. 6a, the electrode 400 may include electrodes having various sizes and shapes. As depicted, the electrode 400 of FIG. 6a may have an electrode pad with a greater size than the electrode 200, 300 of FIGS. 4 and 5. In addition, the electrode 400 of FIG. 6a may have an electrode pad with a different shape from the electrode 200, 300 of FIGS. 4 and 5. By doing so, the TENS apparatus may attach an electrode to various portions of a human body to provide electrical stimulation to the user.

Referring to FIG. 6b, the electrode 500 may be an electrode with a wired cradle shape. In this case, the electrode 500 may be composed of a cradle 510 having a pair of annular contact points, a pair of electrodes 520 and a pair of connection cables 530 for connecting the cradle with each electrode. By doing so, the user may connect the electric signal generator 100 of the present disclosure to an existing cable-type electrode.

Referring to FIG. 6c, the annular contact point 621 of the electrode 600 may be provided at various locations. For example, the annular contact point 621 of the electrode 600 of FIG. 6c may be located at one side of the upper surface of the body, different from the annular contact point of FIGS. 4 and 5 which is located at the center of the upper surface of the body. In this case, as shown in FIG. 6d, the user may easily bend the body of the electrode 600. Therefore, when it is needed to give an electrode signal upwards and downwards, like the wrist, an electrical stimulation suitable for the signal may be provided to the user. In other words, the electrode 600 may be attached to various portions.

The method for operating the TENS apparatus may be implemented as an application or program commands executable by various kinds of computer means and recorded on a computer-readable recording medium. The computer-readable recording medium may include program commands, data files, data structures or the like solely or in combination. The program commands recorded on the medium may be specially designed or configured for the present disclosure or known to and available by computer software engineers.

The computer-readable recording medium includes, for example, magnetic media such as a hard disk, a floppy disk and a magnetic tape, optical media such as CD-ROM and DVD, magneto-optical media such as a floptical disk, hardware devices such as ROM, RAM and a flash memory, specially configured to store and perform program commands, or the like. The program commands include not only machine codes made by a complier but also high-level language codes executable by a computer by using an interpreter. The hardware device may be configured to operate as at least one software module to perform the operations of the present disclosure, or vice versa.

While the exemplary embodiments have been shown and described, it will be understood by those skilled in the art that various changes in form and details may be made thereto without departing from the spirit and scope of this disclosure as defined by the appended claims. In addition, many modifications can be made to adapt a particular situation or material to the teachings of this disclosure without departing from the essential scope thereof.

### MODE FOR THE INVENTION

Various embodiments have been described in the best mode for carrying out the invention.

It will be apparent to those skilled in the art that various modifications and variations can be made in the present invention without departing from the spirit or scope of the invention. Thus, it is intended that the present invention cover the modifications and variations of this invention provided they come within the scope of the appended claims and their equivalents.

### INDUSTRIAL APPLICABILITY

As described above, the present invention is totally or partially applicable to electronic devices.

1. A transcutaneous electrical nerve stimulation (TENS) apparatus, comprising:
an electric signal generator configured to generate an electrical stimulation signal;
an electrode configured to transmit an electrical stimulation corresponding to the electrical stimulation signal generated by the electric signal generator to a user; and
a connection unit configured to connect the electric signal generator to the electrode,
wherein the electrode includes a pair of annular contact points for an electrical connection with the electric signal generator.

2. The TENS apparatus according to claim 1,
wherein the annular contact point is located at a center of an upper surface of the electrode.

3. The TENS apparatus according to claim 1,
wherein the annular contact point is located at one side of an upper surface of the electrode.

4. The TENS apparatus according to claim 1,
wherein the connection unit includes magnetic material.

5. The TENS apparatus according to claim 1,
wherein the electric signal generator includes a pair of output terminals configured to output the electrical stimulation signal, the output terminal being located at a lower surface of a housing.

6. The TENS apparatus according to claim 5,
wherein the electric signal generator further includes an external input terminal configured to receive an input from an external object, the external input terminal being located at one side of the housing.

7. The TENS apparatus according to claim 5, wherein the electric signal generator includes:
a signal generation unit configured to generate the electrical stimulation signal; and
a control unit configured to control each unit of the electric signal generator.

8. The TENS apparatus according to claim 5,
wherein the electric signal generator further includes a communication unit configured to communicate with an external device, in the housing.

9. The TENS apparatus according to claim 1,
wherein the electric signal generator is reusable.

10. The TENS apparatus according to claim 1, wherein the electrode includes:
a body; and
a pair of electrode pads located at a lower surface of the body to transmit the electrical stimulation signal to a user.

11. The TENS apparatus according to claim 1,
wherein the electrode further includes an anti-separation frame configured to preventing the electrode connected to the electric signal generator from being separated therefrom, and the anti-separation frame surrounds the pair of annular contact points.

12. The TENS apparatus according to claim 1,
wherein the electrode is replaceable.

13. The TENS apparatus according to claim 10, wherein the electrode further includes:
a circuit board at which the pair of annular contact points are formed; and
a conductive connection unit connected to the pair of annular contact points to transmit each electrical stimulation signal to the pair of electrode pads.

14. The TENS apparatus according to claim 10,
wherein the body is made of a circuit board at which the annular contact points are formed.

15. The TENS apparatus according to claim 10,
wherein the electrode pad is made of a conductive hydrogel pad which allows the electrode to be attached to the skin of the user.

16. The TENS apparatus according to claim 15,
wherein the conductive hydrogel pad is removable and replaceable.
